(19) United States
(12) Patent Application Publication
(10) Pub. No.: US 2026/0363932 A1
(43) Pub. Date: Nov. 19, 2026

(54) PORTABLE ARTIFICIAL UTERUS

(71) Applicant: CHARITÉ - UNIVERSITÄTSMEDIZIN BERLIN, Berlin (DE)

(72) Inventors: Elisabeth KRENKLER, Berlin (DE); Boris SCHMITT, Berlin (DE)

(73) Assignee: CHARITÉ - UNIVERSITÄTSMEDIZIN BERLIN, Berlin (DE)

(21) Appl. No.: 19/478,091

(22) PCT Filed: Apr. 24, 2024
(86) PCT No.: PCT/EP2024/061227

§ 371 Date: Oct. 23, 2025

(30) Foreign Application Priority Data
Apr. 25, 2023 (DE) 10 2023 110 564.7

(51) Int. Cl. A61G 11/00; A61M 1/16; A61M 1/36

(52) U.S. Cl. CPC A61G 11/00; A61M 1/1698; A61M 1/367; A61M 2209/088; A61M 2240/00

(57) ABSTRACT

It is provided an artificial uterus for caring for a premature baby, in particular until its lungs mature or it reaches full term. The artificial uterus comprises a reservoir with a wall for receiving the premature baby and amniotic fluid. The artificial uterus also comprises an artificial placenta with a connecting piece for connection of the umbilical cord of the premature baby, wherein the artificial placenta comprises at least one membrane via which oxygen from the ambient air around the artificial uterus or from an oxygen reservoir is supplied to the artificial placenta for oxygenating the blood of the premature baby. The artificial uterus furthermore comprises a housing in which the reservoir and the artificial placenta are arranged, wherein the housing in particular has a compact shape. The artificial uterus is portable.

### CROSS-REFERENCE TO A RELATED APPLICATION

This application is a United States national phase Patent Application of International Patent Application Number PCT/EP2024/061227, filed on Apr. 24, 2024, and claims priority of German Patent Application Number 10 2023 110 564.7, filed on Apr. 25, 2023, the disclosure of which are hereby incorporated by reference in their entireties.

### BACKGROUND

This disclosure relates to an artificial uterus for caring for a premature baby, in particular until its lungs mature or it reaches full term. This disclosure furthermore relates to a system for caring for a premature baby, which comprises an artificial uterus.

To care for a premature baby incubators are known, by means of which the premature baby (from today's point of view from a gestational age of 22 weeks) is fed, ventilated, medically cared for, and kept warm. For this purpose, the premature babies are connected to medical devices via a plurality of tubes and probes. Due to the sounds of these devices and the regular interventions of the medical personnel, the premature baby is exposed to physical and psychological stress, whose long-term effects can be assessed only with difficulty. To counteract this, it has become common practice for the premature baby to regularly have close physical contact with its parents (kangaroo care). This physical contact already makes a valuable contribution to the positive development of the premature baby. Nevertheless, the stress factors are maintained.

### SUMMARY

The problem underlying the proposed solution consists in creating a device for caring for a premature baby, which is suitable to also promote the neurological and psychological development of the premature baby beside the physical development and to offer the premature baby an environment which is as similar as possible to the intrauterine environment The device can be used to care for a premature baby for example from a gestational age of 22 weeks up to a gestational age of 28 weeks or possibly 32 weeks.

This problem is solved by providing the artificial uterus with features as described herein.

Accordingly, the artificial uterus comprises a reservoir with a wall for receiving the premature baby and amniotic fluid. Thus, the premature baby in the reservoir can float in amniotic fluid. The amniotic fluid in particular can be artificial amniotic fluid. The environment of amniotic fluid offers a good protection for the sensitive, thin skin of the premature baby. The reservoir in particular mimics the amniotic sac in the womb. Furthermore, the artificial uterus comprises an artificial placenta with a connecting piece for connecting the umbilical cord of the premature baby. The artificial placenta can be arranged on the reservoir (on the outside). The connecting piece can protrude into the reservoir through a lead-through opening of the wall of the reservoir. The connecting piece and the lead-through opening can be connected in a fluid-tight way. The artificial placenta comprises at least one membrane via which oxygen (for example from the ambient air around the artificial uterus and/or from an oxygen reservoir which stores compressed oxygen) is supplied to the artificial placenta for oxygenating the blood of the premature baby. The use of the at least one membrane hence provides for supplying oxygen from the ambient air around the artificial uterus (and/or from the oxygen reservoir) for oxygenating the blood of the premature baby. The use of the at least one membrane hence provides for utilizing oxygen from the ambient air around the artificial uterus and/or from the oxygen reservoir. When the oxygen from the ambient air is sufficient, the storage of (pure) oxygen in corresponding gas cylinders can be omitted. Moreover, the at least one membrane can serve to discharge carbon dioxide from the artificial placenta. To support the circulation and the supply of the ambient air (and the discharge of the carbon dioxide produced), one or more fans can be provided for a better circulation of air. The reservoir and the artificial placenta are arranged in a housing of the artificial uterus. The housing in particular has a compact shape. Compact shape is understood to be a shape in which the ratio of surface area of the housing to its volume is as small as possible, with the objective that the artificial uterus can be grasped and held with two hands and/or can be carried on the body of a user by means of a carrying system. The ratio of surface area of the housing to its volume need not be minimal, so that the housing for example can have a flat side or a flat portion. On the one hand due to the compact shape of the housing and on the other hand due to the membrane, the artificial uterus is portable in a functional condition. The artificial uterus is an autonomous, mobile system closed in itself. This means that it can be used independently of tube- and/or cable-connected immobile devices (for example for the ventilation or feeding of the premature baby). This allows the user to carry the device extremely close to their body and enjoy maximum mobility, so that the user in particular can also leave the hospital with the premature baby in the artificial uterus. The mobility of the user can merely be restricted by the need to be close to the treating, monitoring hospital or physician and by the possibility of being reachable via a telephone network.

A carrying system can be attached or attachable to the housing. The connection between the housing and the carrying system can be a separable connection. Alternatively or additionally, it can be provided to accommodate the housing in a receptacle of a carrying system. The receptacle for example can be of pocket-like design. The housing can thus be carried by means of the carrying system. The carrying system is provided for attachment to an upper part of the body of a user. Thus, the artificial uterus can be carried by the user. In particular, the artificial uterus can be carried in front of the belly of the user. For example, the parents of the premature baby can carry the artificial uterus in front of their belly by means of the carrying system. The premature baby thus experiences the movement and sounds of the parents. This allows the premature baby to experience conditions similar to those in the intrauterine environment and promotes the neurological and psychological development of the premature baby. According to one embodiment, the carrying system comprises a lap belt and two shoulder straps. Thus, the weight of the artificial uterus, of the premature baby located therein and of the amniotic fluid can be distributed in a way which is as gentle on the user's back as possible.

According to one embodiment, the wall of the reservoir largely completely encloses a volume. Hence, the wall defines an interior space of the reservoir in which the amniotic fluid is accommodated. In particular, the wall is such that the amniotic fluid cannot exit from the reservoir in an uncontrolled way. In the wall, at least one (closable) opening can be provided, through which protrudes for example a (closable) lead-through. The at least one opening or lead-through provides for an access to the interior space of the reservoir.

For example, an access port can be formed in the wall of the reservoir or in the opening of the wall, via which samples of amniotic fluid can be taken. This or a further access port can be provided in order to exchange the amniotic fluid. There can also be provided two access ports in order to on the one hand discharge amniotic fluid from the reservoir and on the other hand introduce amniotic fluid into the reservoir. For this purpose a pump can be provided, which is configured to (continuously) pump the amniotic fluid out of the reservoir and again pump it into the same. Outside the reservoir a device for cleaning the amniotic fluid can be provided. This device can comprise filters for the purpose of cleaning the amniotic fluid and/or provide UV radiation. The amniotic fluid discharged can thus be cleaned and again be supplied (directly) to the reservoir after cleaning. Alternatively, the amniotic fluid can be exchanged completely.

It is conceivable that the wall of the reservoir is made of a stretchable material, in particular latex. Thus, the volume of the reservoir (similar to the amniotic sac in the womb) can grow with the premature baby. Furthermore, it is conceivable to adjust the size/the volume of the reservoir by means of a device. For example, the device can comprise a multi-part clamp which extends around the reservoir. Depending on the desired volume of the reservoir, the number of the links of the clamp can be selected. To for example increase the volume of the reservoir, the number of the clamp links is increased correspondingly. It is also conceivable that the device comprises a plurality of (concentrically arranged) rings of different diameters, which extend around the reservoir. To provide for an increase of the volume of the reservoir, individual rings can be removed one after the other (with increasing diameter). Along with the growth of the premature baby in the reservoir, the pressure inside the reservoir also rises. For example, on the basis of (regular) pressure measurements the time can be determined, at which the device is to be changed (to add clamp links or to remove rings) in such a way that the volume of the reservoir can increase.

According to one embodiment, the housing can be extendable and/or have a variable volume. For example, the housing can comprise a plurality of modules. There can be provided a base module and a number of add-on modules, wherein the add-on modules have different sizes and are provided for attachment to the base module. The base module can each be combined with one of the add-on modules, in order to form the housing. Depending on the choice of the add-on module, the size/the volume of the housing can be set. It is also conceivable that an adjusting mechanism is provided, by means of which the volume of the housing can be varied. In this context, the housing sectionally can include several (at least two) overlapping material layers, wherein the extent of their overlap is variable and can be set by means of the adjusting mechanism. It is also conceivable that the material of the housing has at least one fold for example in defined areas. Depending on the desired volume of the housing, the folded areas can (completely or partly) be unfolded by means of the adjusting mechanism.

The shape of the housing can be such that it imitates the belly of a pregnant woman. The housing can comprise a flat portion and a dome-like portion which extends over the flat portion. The flat portion and the dome-like portion thus enclose a housing volume. When the artificial uterus is carried on the upper part of the body of a user, the flat portion can rest against the belly of this user or face the belly of this user. The above-mentioned base module for example can form or comprise the flat portion. The above-mentioned add-on module for example can form or comprise the dome-like portion.

According to another embodiment, the housing is impact-resistant. The premature baby thus can be protected from impacts. On the inside of the housing, which faces the reservoir, there can also be provided a padding in order to absorb impacts of the reservoir against the housing on movement of the artificial uterus.

Furthermore, the artificial uterus can comprise a tempering device which is configured to maintain a predetermined temperature in the interior space of the reservoir. In particular, the predetermined temperature can lie in a range between 36°C and 38 °C. The tempering device can comprise a temperature measuring device and a heating device as well as a control unit which controls the heating device in dependence on the measurement data of the temperature measuring device. For operating the tempering device, a rechargeable energy accumulator can be provided. The rechargeable energy accumulator can be arranged in the housing. Between the reservoir and the housing an insulation layer can at least sectionally be provided for thermal insulation. For example, the insulation layer is provided between the dome-like portion of the housing and the reservoir, while between the flat portion of the housing and the reservoir no insulation layer is provided. Alternatively, the insulation layer can enclose the entire reservoir.

For monitoring the state of the premature baby various sensors can be provided. For example, there can be provided at least one sensor for monitoring the oxygen saturation of the blood of the premature baby and/or at least one sensor for monitoring the heart rate of the premature baby. The sensors can be arranged on the artificial placenta, near the umbilical cord of the premature baby. The sensors can be supplied with energy by means of the rechargeable energy accumulator mentioned already. The measurement data can be (wirelessly) transmitted to a monitoring device. For taking blood samples of the premature baby a blood sampling faucet (port) can be provided on the placenta, near the umbilical cord of the premature baby.

For the purpose of feeding the premature baby, the artificial uterus can comprise a feeding device which includes a storage container for nutrition and a pump. The pump is configured to pump the nutrition from the storage container into the umbilical cord of the premature baby or into the connecting piece of the placenta. The pump therefor includes the required connecting elements. The pump can be controlled by means of a pump controller. The pump controller for example is configured to control the pump at regular intervals, in dependence on a measurement signal, or as a result of an input of a user. The pump and the pump controller can also be supplied with energy by means of the rechargeable energy accumulator mentioned already. The storage container can include a port so that the storage container can be filled when necessary. Alternatively, the storage container can be designed as a disposable container so that an emptied storage container is replaced by a new, filled storage container.

The solution furthermore relates to a system for caring for a premature baby. First of all, this system comprises an artificial uterus of the type presented here. Furthermore, the system comprises a monitoring device which is configured to receive and evaluate measurement data from sensors of the artificial uterus (sensor for monitoring the oxygen saturation of the blood of the premature baby, sensor for monitoring the heart rate of the premature baby). The measurement data for example are the oxygen saturation of the blood of the premature baby or the heart rate of the premature baby. Further (continuously detected) measurement data from the examination of the blood of the premature baby for example are the oxygen partial pressure (p_{O2}), carbon dioxide partial pressure (p_{CO2}), carbon dioxide (CO₂), glucose content, pH value, base excess (BE), electrolyte content (sodium, potassium, calcium, chloride, fluoride, anion gap), content of trace elements (zinc, iron). Furthermore, the total protein value, the concentration of interleukin-6 (IL-6) in plasma, and the CRP concentration (C-reactive protein) in plasma can be measured externally (e.g., once a day) in a blood sample taken from the premature baby using a point-of-care (POC) diagnostic device. The monitoring device can receive the measurement data in particular wirelessly from the sensors. The monitoring device can be accommodated for example in a hospital and/or in a doctor's office. The monitoring device can be configured to generate a signal in dependence on the measurement data. To display the signal to a user, the system can comprise at least one display device. In principle, several display devices can be provided. For example one display device can likewise be accommodated in the hospital and/or the doctor's office and serve the medical personnel for the close/permanent monitoring of the premature baby (from a distance). Further display devices for example can be provided for the user of the artificial uterus, in particular in the form of portable terminal devices (smart phone, smart watch, etc.). The signal which is displayed on a display device intended for a medical layperson can differ in complexity from the signal displayed on another display device intended for medical personnel. Thus, for example based on a synopsis of all measurement data either a first signal can be displayed for the medical layperson, which signal represents the inconspicuous state of the premature baby, or a second signal which corresponds to a request to seek or contact medical personnel. On the other hand, the signal which is provided for the medical personnel can comprise all details of the measurement data. It can thereby be achieved that the medical layperson is not confused by fluctuations in the measurement data and merely is informed when there is an actual need for action. At the same time, the medical personnel able to interpret the measurement data professionally has access to all the information needed to make decisions about measures to be taken based on comprehensive knowledge. It is also conceivable that the monitoring device comprises an input medium which for example is available to the medical personnel. The input medium can serve to enter information or instructions of the medical personnel. On the display device, which is available to the user of the artificial uterus, the information or instructions of the medical personnel can be displayed.

The system can comprise a base station for stocking the artificial uterus. The base station can have a shape which (at least sectionally) is complementary to the flat portion of the housing. In particular, the base station can include a charging device for charging the energy accumulator of the artificial uterus. It is also conceivable that the base station comprises a tempering device which with respect to construction and function corresponds to the tempering device of the artificial uterus. A sensor can be provided, in order to detect whether the artificial uterus is properly arranged at/on the base station. The measurement data can serve to control the two tempering devices. In the positive case, it can be provided that (exclusively) the tempering device of the base station is in operation, whereas in the negative case (exclusively) the tempering device of the artificial uterus is in operation.

### BRIEF DESCRIPTION OF THE DRAWINGS

The solution will subsequently be explained in detail by means of exemplary embodiments with reference to the Figures.

FIGS. 1-5 show various views of an artificial uterus according to one embodiment.

FIG. 6 shows a schematic representation of the care for the premature baby by means of the artificial uterus from FIGS. 1 to 5.

FIG. 7 shows a front view of a user with the artificial uterus from FIGS. 1 to 5 together with a carrying system.

FIG. 8 shows a rear view of the user from FIG. 7 with the carrying system.

FIG. 9 shows the artificial uterus from FIGS. 1 to 5 together with a base station.

FIG. 10 shows a schematic representation of a system for caring for a premature baby according to one embodiment.

### DETAILED DESCRIPTION

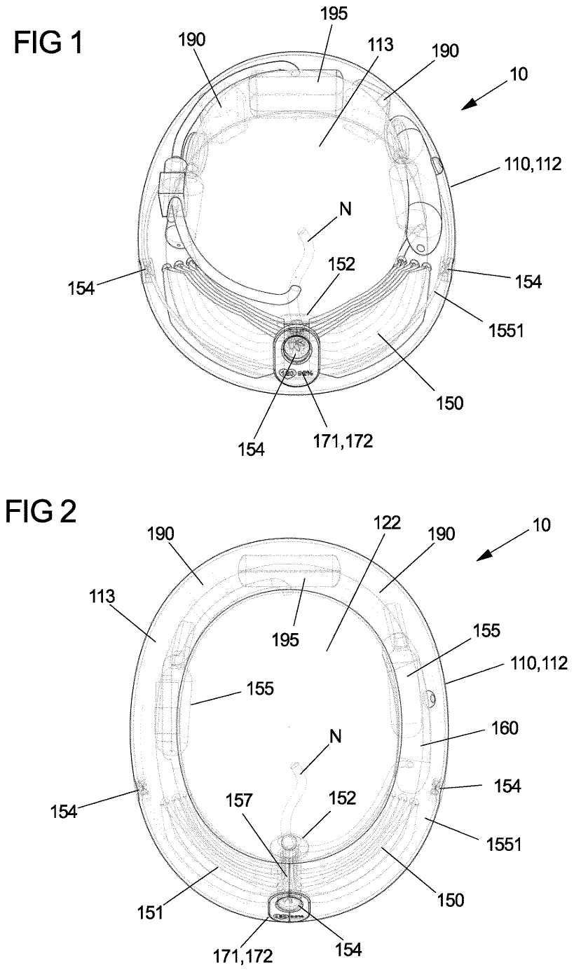

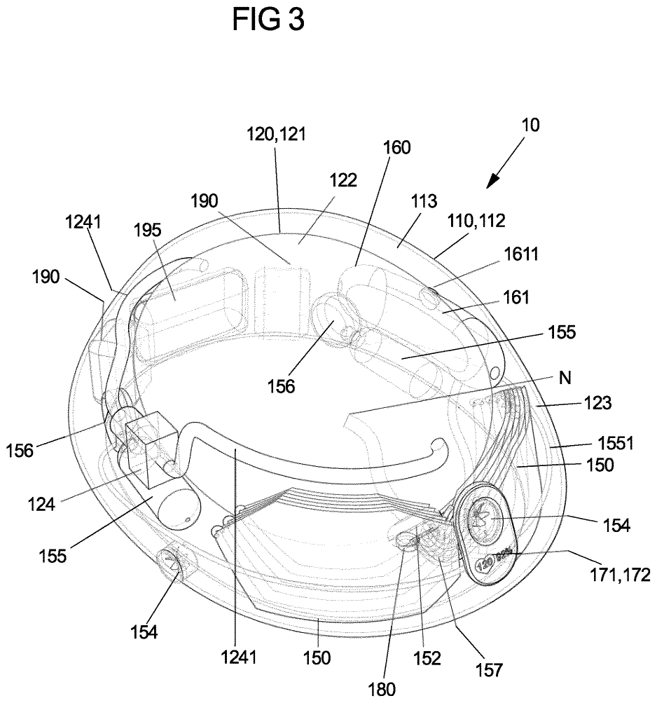

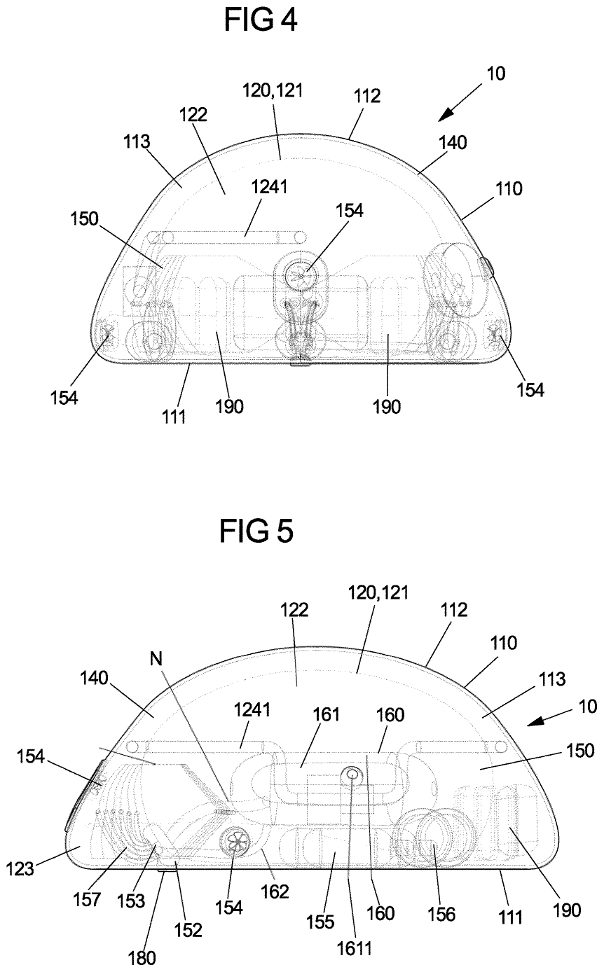

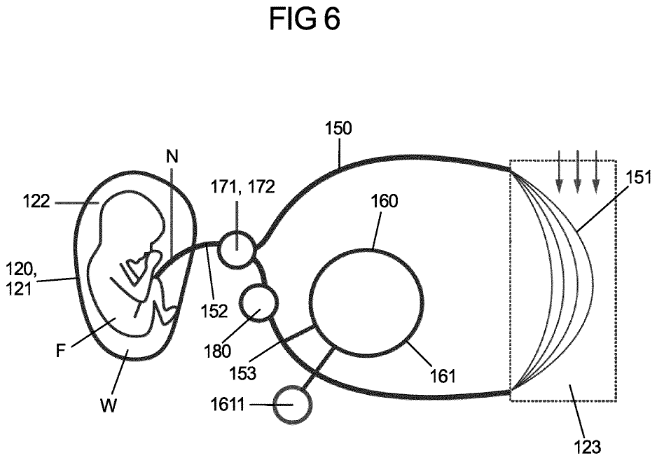

FIGS. 1 to 5 show an artificial uterus 10 according to one embodiment of the solution in various perspectives. FIG. 6 schematically shows the care for a premature baby F in this artificial uterus 10. The artificial uterus 10 comprises a housing 110 which defines the outer shape of the artificial uterus 10. The housing 110 comprises a flat portion 111 and a dome-like portion 112 which extends above the flat portion 111. The flat portion 111 and the dome-like portion 112 together enclose a housing volume 113.

In the housing 110 a reservoir 120 is arranged for receiving the premature baby F and amniotic fluid W. The reservoir 120 comprises a wall 121 which (almost) completely encloses an interior space 122 of the reservoir 120.

For continuously cleaning the amniotic fluid W a device 124 for cleaning the amniotic fluid is provided, which comprises a pump as well as filters and a UV radiation source. By means of the pump, the amniotic fluid W present in the reservoir 120 is pumped continuously to the device 124 for the purpose of cleaning, and the amniotic fluid W cleaned by the device 124 is again pumped from there into the reservoir 120. Between the reservoir 120 and the device 124, corresponding lines 1241 are formed for conducting amniotic fluid W.

Between the reservoir 120 and the housing 110 a tempering device (not shown) is provided, which is configured to maintain the temperature in the interior space 122 of the reservoir 120 in a predetermined range. The tempering device comprises a heating device and a temperature measuring device as well as a control unit which controls the heating device in dependence on the measurement data of the temperature measuring device. The heating device extends for example in a net-like manner around the entire reservoir 120. Alternatively, the heating device can be configured to guide tempered air into the space between the reservoir 120 and the housing 110 and thus maintain the temperature in the interior space 122 of the reservoir 120 in a predetermined range. The temperature measuring device can carry out the temperature measurement at several points on the wall 121 of the reservoir 120.

Between the reservoir 120 and the housing 110 there is furthermore provided an insulation layer 140 for the thermal insulation of the reservoir 120. The insulation layer 140 for example surrounds the heating device of the tempering device and directly adjoins the heating device.

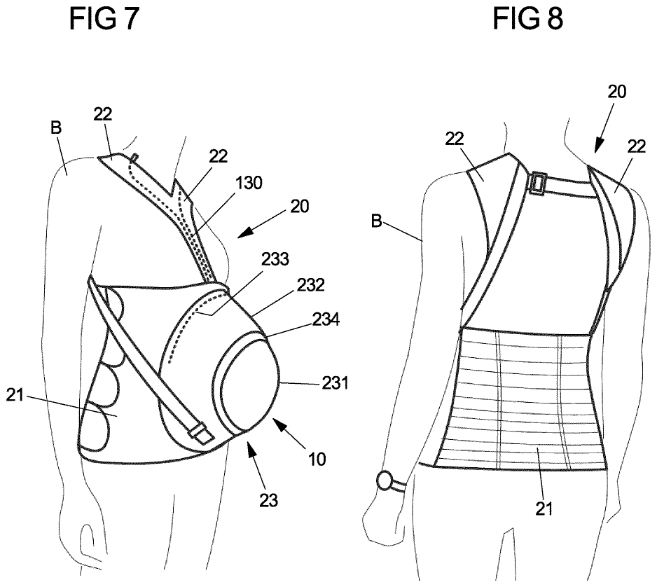

In the housing 110 there is furthermore provided an artificial placenta 150 for supplying the premature baby F with oxygen (and nutrition). The artificial placenta 150 comprises membranes 151 via which oxygen from the ambient air around the artificial uterus 10 is supplied to the artificial placenta 150 for oxygenating the blood of the premature baby F. In particular, the ambient air can be air which is present in the surroundings of the membranes 151 within the housing 110 (in an air chamber 123). To improve the circulation of the ambient air in the region of the membranes 151, at least one fan 154 (here by way of example three fans 154) is provided. In the embodiment of FIGS. 1 to 5 the fans 154 on the one hand are provided in the region of the membranes 151 in which the membranes 151 absorb the oxygen, and on the other hand in the region of the membranes 151 in which the membranes 151 release carbon dioxide. To provide for the supply of air also under the clothing of a user B, tubes 130 for example are provided, which lead into the vicinity of the fans 154 and thus supply ambient air to the fans 154 or discharge air richer in carbon dioxide from the air chamber 123 into the environment of the artificial uterus 10. These tubes 130 (as shown by way of example in FIG. 7) can be discreetly attached to an edge of the clothing, for example at the neckline, by means of a clip.

Additionally or alternatively to the ambient air, the required oxygen can also be obtained from an oxygen reservoir 155. The oxygen reservoir 155 can be arranged in the housing 110. The oxygen reservoir 155 cooperates with a valve 156 or a similar device, in order to regulate the pressure of the oxygen escaping from the oxygen reservoir 155. Via a line 1551, the oxygen is conducted from the oxygen reservoir 155 to a region of the membrane 151 in which the membranes 151 absorb the oxygen. There can also be provided a control unit which controls the valve 156 in dependence on a regularly detected measured value. The measured value for example can be the oxygen saturation of the blood of the premature baby For the oxygen concentration in the air chamber 123.

A feeding device 160 cooperates with the artificial placenta 150. The feeding device 160 comprises a storage container 161 for nutrition and a pump which pumps the nutrition from the storage container 161 via a line 162 into the artificial placenta 150 or to a connecting piece 152 described below. The artificial placenta 150 therefor includes a corresponding access port 153 for nutrition. For filling the storage container 161 the same includes a port 1611.

The artificial placenta 150 is connected to the umbilical cord N of the premature baby F via the connecting piece 152. Between the connecting piece 152 and the membranes 151 lines 157 are provided for the transport of oxygen-poor blood from the umbilical cord N to the membranes 151 and of oxygen-rich blood from the membranes 151 to the umbilical cord N. The connecting piece 152 can be formed with a pumping device which supports the corresponding blood flow. The blood enriched with oxygen and supplied with nutrition flows into the umbilical cord N of the premature baby F via the connecting piece 152 of the artificial placenta 150.

In the transition region between the artificial placenta 150 and the umbilical cord N of the premature baby F sensors 171, 172 are formed for monitoring the heart rate and the oxygen saturation of the blood of the premature baby F. Likewise in this region, there is also located a port 180 for taking blood samples.

For supplying the device 124 for cleaning the amniotic fluid, the tempering device, the pump of the feeding device 160, the sensors 171, 172, valves 156 and fans 154 (and possible further energy-consuming components) with energy a chargeable energy accumulator 190 is provided.

The device 124 for cleaning the amniotic fluid, the tempering device, the pump of the feeding device 160, all sensors 171, 172, valves 156 and fans 154 are controlled by a control unit 195. The control unit 195 can likewise be arranged in the housing 110.

In FIGS. 7 and 8, the housing 110 of the artificial uterus 10 is arranged in a carrying system 20 which serves to attach the artificial uterus 10 to the upper part of the body of a user B. The carrying system 20 comprises a lap belt 21 and two shoulder straps 22 as well as a receptacle 23. The receptacle 23 is attached to the lap belt 21 and the shoulder straps 22 and can be carried on the belly of the user B. The receptacle 23 serves to receive the housing 110 of the artificial uterus 10. The receptacle mimics the shape of the housing 110 and in particular is shaped similar to a hemisphere. The receptacle 23 comprises an inner material layer 231 and an outer material layer 232 at least sectionally surrounding the inner material layer 231. While the outer material layer 232 is made of a solid, dimensionally stable material/fabric, the inner material layer 231 is made of a relatively stretchable material/fabric. It is provided that the artificial uterus 10 is arranged in the inner material layer 231 of the receptacle 23. To provide for access to the interior space of the receptacle 23, the receptacle 23 includes an opening mechanism 233. The opening mechanism 233 can assume an open configuration and a closed configuration. In the open configuration it is possible to place the artificial uterus 10 (and the inner material layer 231) in the receptacle 23 (in the outer material layer 232) or to again remove it from the receptacle 23 (in the outer material layer 232). In the closed configuration, the artificial uterus 10 is safely accommodated in the receptacle 23 and protected from slipping out inadvertently. The opening mechanism 233 in FIG. 7 for example is formed as a zipper.

In the embodiment of FIG. 7, the outer material layer 232 only sectionally covers the inner material layer 231. The outer material layer has a shape which comes close to a truncated cone. On the (annular) edge of the outer material layer 232, which forms the boundary between the covered portion of the inner material layer 231 and the uncovered portion of the inner material layer, the outer material layer 232 is provided with a size-adjustable element 234. The size-adjustable element 234 is provided to adjust the size of the outer material layer 232, in particular in the region of said edge. For example, the size-adjustable element 234 can be of cord-like design and can be arranged in an (annular) sleeve of the outer material layer 232 following said edge. By a change in length (shortening) of the cord-like element 234, the size of the outer material layer 232 can be adjusted (by gathering).

In another embodiment (not shown), the housing 110 can be releasably attached to the lap belt 21.

The carrying system 20 provides for carrying the artificial uterus 10 so as to rest against the belly of the user B. FIG. 7 shows that the artificial uterus 10 rests against the belly of the user B with the flat portion 111 of the housing 110.

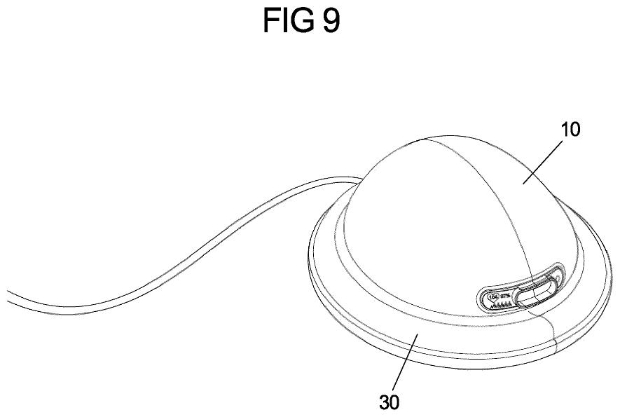

The artificial uterus 10 presented in FIGS. 1 to 5 is part of a system 1 for caring for a premature baby F. Beside the artificial uterus 10, the system 1 comprises a base station 30 (FIG. 9). The artificial uterus 10 can be deposited on the base station 30 when it is not carried by a user B by means of the carrying system 20. The base station 30 can be configured to charge the chargeable energy accumulator 190 of the artificial uterus 10 when the artificial uterus 10 is properly arranged at/on the base station 30.

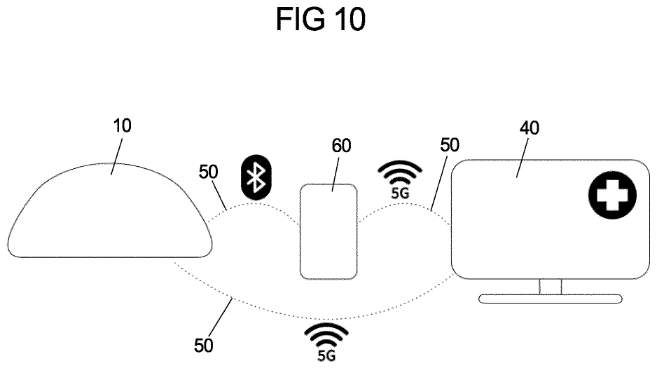

Furthermore, the system 1 comprises a monitoring device 40 which receives and evaluates the measurement data of the sensors 171, 172 (FIG. 10). The measurement data are transmitted from the sensors 171, 172 of the artificial uterus 10 to the monitoring device 40 via a wireless communication connection 50. Therefore, the monitoring device 40 can be spatially separated from the artificial uterus 10. The monitoring device 40 for example can be located in a hospital, while the artificial uterus 10 is located with the parents of the premature baby F at home or elsewhere outside the hospital. The monitoring device 40 is configured to generate a signal on the basis of the evaluated measurement data and send this signal to a display device 60. The display device 60 comprises several display elements, which are available on the one hand for the user B of the artificial uterus 10 and on the other hand for medical personnel. In FIG. 10, the display device 10 likewise is connected to the sensors 171, 172 and to the monitoring device 40 via a wireless communication connection 50. The display device 60 can also be connected to the monitoring device 40 only. The types of the wireless communication shown in FIG. 10 merely are by way of example.

1. An artificial uterus for caring for a premature baby, comprising
a reservoir with a wall for receiving the premature baby and amniotic fluid;
an artificial placenta comprising a connecting piece for connection of the umbilical cord of the premature baby, wherein the artificial placenta comprises at least one membrane via which oxygen from the ambient air around the artificial uterus or from an oxygen reservoir is supplied to the artificial placenta for oxygenating the blood of the premature baby; and
a housing in which the reservoir and the artificial placenta are arranged, wherein the housing,
wherein the artificial uterus is portable.

2. The artificial uterus according to claim 1, further comprising a carrying system which can be attached to the housing or in which the housing can be arranged and which is provided for attachment to an upper part of the body of a user, in order to carry the artificial uterus.

3. The artificial uterus according to claim 2, wherein the carrying system comprises a lap belt and two shoulder straps.

4. The artificial uterus according to claim 1, wherein the wall of the reservoir completely encloses an interior space of the reservoir.

5. The artificial uterus according to claim 1, wherein the wall of the reservoir is made of a stretchable material, so that the volume of the reservoir can grow with the premature baby.

6. The artificial uterus according to claim 1, wherein the housing is expandable and/or has a variable volume.

7. The artificial uterus according to claim 1, wherein the housing is impact-resistant.

8. The artificial uterus according to claim 1, wherein the housing comprises a flat portion and a dome-like portion which extends over the flat portion, so that the flat portion and the dome-like portion include a housing volume.

9. The artificial uterus according to claim 1, further comprising a tempering device which is configured to maintain a predetermined temperature inside the wall of the reservoir.

10. The artificial uterus according to claim 1, wherein between the reservoir and the housing an insulation layer is provided for thermal insulation.

11. The artificial uterus according to claim 1, further comprising at least one sensor for monitoring the oxygen saturation of the blood of the premature baby and/or at least one sensor for monitoring the heart rate of the premature baby.

12. The artificial uterus according to claim 1, wherein an access port is formed in the artificial placenta, via which samples of blood of the premature baby can be taken.

13. The artificial uterus according claim 1, further comprising a feeding device comprising a storage container for nutrition and a pump which is configured to pump the nutrition from the storage container into the umbilical cord of the premature baby or into the connecting piece of the artificial placenta.

14. The artificial uterus according to claim 13, wherein a pump controller is provided, which is configured to control the pump at regular intervals in dependence on a measurement signal or as a result of an input of a user.

15. A system for caring for a premature baby, comprising an artificial uterus according to claim 1 and a monitoring device, wherein the monitoring device is configured to receive and evaluate measurement data relating to the artificial uterus.

16. The system for caring for a premature baby according to claim 15, wherein the monitoring device generates a signal in dependence on the measurement data.

17. The system for caring for a premature baby according to claim 16, further comprising a display device which is configured to display the signal generated in dependence on the measurement data to a user.

18. The system according to claim 15, further comprising a base station for stocking the artificial uterus.

19. The system according to claim 18, wherein the base station includes a charging device for charging at least one energy accumulator of the artificial uterus.
